(19) United States
(12) Patent Application Publication
(10) Pub. No.: US 2023/0201313 A1
(43) Pub. Date: Jun. 29, 2023

(54) METHODS OF TREATING LEFT VENTRICLE HYPERTROPHY

(71) Applicants: Amgen Inc., Thousand Oaks, CA (US); Medical University of Vienna, Vienna (AT)

(72) Inventors: Rainer OBERBAUER, Vienna (AT); Bruno FOUQUERAY, Risch-Rotkreuz (CH); Katharina DÖRR, Vienna (AT)

(21) Appl. No.: 17/925,581

(22) PCT Filed: May 14, 2021
(86) PCT No.: PCT/US2021/032597

§ 371 Date: Nov. 15, 2022

Related U.S. Application Data
(60) Provisional application No. 63/025,626, filed on May 15, 2020.

(51) Int. Cl. A61K 38/29; A61P 5/20; A61P 9/00

(52) U.S. Cl. CPC A61K 38/29; A61P 5/20; A61P 9/00

(57) ABSTRACT

Disclosed are methods for treatment of a subject with left ventricle hypertrophy. Also disclosed are methods for slowing or delaying progression of left ventricle hypertrophy, as well as methods for mediating cardiac remodeling and for improving cardiac function in subjects with left ventricle hypertrophy. The methods comprise administering etelcalcetide parenterally to a subject.

### CROSS-REFERENCE TO RELATED APPLICATIONS

This claims priority to U.S. Provisional Patent Application No. 63/025,626, filed on May 15, 2020, the entire content of which is incorporated by reference herein.

### TECHNICAL FIELD

The subject matter described herein relates to a method of treating left ventricle hypertrophy and for slowing progression of left ventricle hypertrophy in a subject on chronic hemodialysis by administering etelcalcetide.

### BACKGROUND

Persons with chronic kidney disease (CKD) develop left ventricular hypertrophy (LVH) and cardiac fibrosis which contributes to congestive heart failure, diastolic dysfunction, arrhythmia and sudden death (Di Marco, G. S. et al., Nephrology Dialysis Transplantation, 2014: p. 29:2028-34; Faul, C. et al., J Clin. Invest., 2011: p. 121:4393-408; London, G. M. et al., Am. Soc. Nephrol., 2001: p. 12:2759-67). The majority of patients with terminal renal failure treated by dialysis exhibit LVH and have a dramatically increased risk of sudden cardiac death (Foley, P. P et al., Kidney International, 1995: p. 47:186-92).

Main drivers of cardiac remodeling in hemodialysis patients are chronic volume overload, intradialytic weight gain and hemodynamic fluctuations during hemodialysis treatment (AM, K., Cardiovasc Drugs Ther., 2002: p. 16:245-9; Salerno, M. P. et al., Transplantation Proceedings, 2013: p. 45:2660-2). Additional factors include elevated fibroblast growth factor 23 (FGF23) levels in CKD and dialysis patients and angiotensin II mediated cardiac remodeling (Brilla, P. R et al., Circ Res., 1990: p. 67:1355-64; Brilla, P. R et al., Eur Heart J, 1995: p. 16:107-9). Circulating concentrations of FGF23 increase progressively as the glomerular filtration rate declines beginning as early as in CKD stage 3b (Wolf, M., et al., Journal of the American Society of Nephrology, 2011: p. 22:956-66; Shigematsu T, K. J et al., American Journal of Kidney Diseases, 2004: p. 44:250-6). The left ventricular mass index (LVMI) rises with increasing FGF23 and so does the prevalence of eccentric and concentric hypertrophy (Faul, C. et al., J. Clin. Invest., 2011: p. 121:4393-408). The pathophysiological mechanism by which FGF23 may cause LVH is not well understood and it remains unknown whether treatment with the calcimimetic etelcalcetide, which also lowers FGF23 levels, reduces or retards the development of LVH in patients on hemodialysis.

The foregoing examples of the related art and limitations related therewith are intended to be illustrative and not exclusive. Other limitations of the related art will become apparent to those of skill in the art upon a reading of the specification and a study of the drawings.

### BRIEF SUMMARY

The following aspects and embodiments thereof described and illustrated below are meant to be exemplary and illustrative, not limiting in scope.

In one aspect, a method of treatment for a subject with left ventricle hypertrophy (LVH) is provided. The method comprises administering etelcalcetide to alleviate progression of LVH.

In an embodiment, alleviating progression of LVH treats the subject.

In one embodiment, etelcalcetide is administered parenterally.

In one embodiment, etelcalcetide is administered intravenously.

In another embodiment, etelcalcetide is administered subcutaneously.

In one embodiment, the subject is a subject on chronic hemodialysis.

In one embodiment, the subject receives hemodialysis two times per week. In another embodiment, the subject receives hemodialysis three times per week.

In another embodiment, the subject has received hemodialysis approximately three times per week for at least about 3 months.

In another embodiment, the subject is a subject undergoing peritoneal dialysis.

In one embodiment, the subject is pre-dialysis.

In some embodiments, administering is for a period of 12 months. In other embodiments, administering is for a period of 12 months and is effective to alleviate progression, slow progression, and/or delay progression of left ventricle hypertrophy by a median of about 6.0 g/m², about 6.3 g/m², about 6.7 g/m² or about 7.0 g/m² relative to left ventricle hypertrophy before the administering.

In another aspect, a method for slowing progression of left ventricle hypertrophy in a subject on chronic dialysis and with left ventricle hypertrophy is provided. The method comprises administering etelcalcetide, whereby the administering slows progression of left ventricle hypertrophy.

In one embodiment, the subject is one on chronic hemodialysis. In another embodiment, the subject is one on chronic peritoneal dialysis.

In one embodiment, etelcalcetide is administered intravenously. In another embodiment, etelcalcetide is administered subcutaneously.

In one embodiment, administering slows progression of left ventricle hypertrophy as measured by left ventricular mass index through cardiac magnetic resonance imaging (cMRI) by a median of about 6.0 g/m², about 6.3 g/m², about 6.7 g/m² or about 7.0 g/m² relative to the subject's left ventricle hypertrophy mass index before the administering.

In another aspect, a method for delaying progression of left ventricle hypertrophy in a subject on chronic dialysis and with left ventricle hypertrophy is provided. The method comprises administering etelcalcetide before, during or after dialysis for at least approximately 12 months.

In one embodiment, the subject is one on chronic hemodialysis. In another embodiment, the subject is one on chronic peritoneal dialysis.

In one embodiment, etelcalcetide is administered intravenously. In another embodiment, etelcalcetide is administered via a catheter.

In one embodiment, administering delays progression of left ventricle hypertrophy, as measured by left ventricular mass index through cMRI, by a median of about 6.0 g/m², about 6.3 g/m², about 6.7 g/m² or about 7.0 g/m² relative to left ventricle hypertrophy mass index in a population of subjects on maintenance hemodialysis and with left ventricle hypertrophy not yet treated with etelcalcetide.

In an embodiment of any of the aspects, administering alleviates progression of left ventricle hypertrophy, as measured by left ventricular mass index through cardiac magnetic resonance imaging (cMRI).

In another aspect, a method to mediate cardiac remodeling in a subject with left ventricle hypertrophy and on chronic dialysis is provided. The method comprises administering etelcalcetide intravenously before, during or after dialysis for at least approximately 12 months.

In one embodiment, the subject is one on chronic hemodialysis. In another embodiment, the subject is one on chronic peritoneal dialysis.

In one embodiment, etelcalcetide is administered intravenously. In another embodiment, etelcalcetide is administered subcutaneously.

In an embodiment, the administering reduces left ventricle hypertrophy as determined by a cMRI assessment of left ventricular mass index by a median of about 6.0 g/m², about 6.3 g/m², about 6.7 g/m² or about 7.0 g/m² relative to left ventricle hypertrophy mass index in a population of subjects on maintenance hemodialysis and with left ventricle hypertrophy not yet treated with etelcalcetide.

In an embodiment, the subject receives hemodialysis two times, three times or four times per week.

In an embodiment of any of the aspects, the subject has received hemodialysis three times per week for at least about 3 months.

In an embodiment of any of the aspects, the subject has secondary hyperparathyroidism (sHPT).

In an embodiment of any of the aspects, the subject has a parathyroid hormone (PTH) level of greater than or equal to 300 pg/mL prior to administering etelcalcetide.

In an embodiment of any of the aspects, the administering is at a dose of etelcalcetide that provides a blood parathyroid hormone (PTH) level of less than about 300 pg/mL.

In an embodiment of any of the aspects, administering is at a dose of etelcalcetide that provides a blood parathyroid hormone (PTH) level of between about 100 pg/mL to about 300 pg/mL.

In an embodiment of any of the aspects, the administering slows development of cardiac fibrosis.

In another aspect, a method to improve cardiac function in a subject with left ventricle hypertrophy and on maintenance hemodialysis is provided. The method comprises reducing circulating blood FGF23 levels in the subject for at least about 6 months by administering etelcalcetide intravenously subsequent to hemodialysis for at least about 6 months.

In another aspect, a method to improve cardiac function in a subject with left ventricle hypertrophy and on maintenance dialysis is provided. The method comprises reducing circulating blood FGF23 levels in the subject for at least about 6 months by administering etelcalcetide before, during or after dialysis for at least about 6 months.

In one embodiment, the subject is one on chronic hemodialysis. In another embodiment, the subject is one on chronic peritoneal dialysis.

In one embodiment, the administering slows development of cardiac fibrosis as measured by T1 weighted cMRI.

In another embodiment, the administering is for at least about 9 months or for at least about 12 months.

In another aspect, a method to reduce likelihood of cardiac death in a subject with left ventricle hypertrophy on maintenance hemodialysis is provided. The method comprises reducing circulating blood FGF23 levels in the subject for at least about 6 months by administering etelcalcetide intravenously subsequent to hemodialysis for at least about 6 months.

In one embodiment, the administering slows development of cardiac fibrosis as measured by T1 weighted cMRI.

In another embodiment, the administering is for at least about 9 months or for at least about 12 months.

In addition to the exemplary aspects and embodiments described above, further aspects and embodiments will become apparent by reference to the drawings and by study of the following descriptions.

Additional embodiments of the present methods, and the like, will be apparent from the following description, drawings, examples, and claims. As can be appreciated from the foregoing and following description, each and every feature described herein, and each and every combination of two or more of such features, is included within the scope of the present disclosure provided that the features included in such a combination are not mutually inconsistent. In addition, any feature or combination of features may be specifically excluded from any embodiment of the present disclosure. Additional aspects and advantages of the present disclosure are set forth in the following description and claims, particularly when considered in conjunction with the accompanying examples and drawings.

### BRIEF DESCRIPTION OF THE DRAWINGS

FIG. 1A is an illustration of the study conducted herein.

FIGS. 1B-1C show the timeline of the planned procedures, study visits and scheduled dose titrations of the study of Example 1.

FIGS. 2A-2B are boxplots of unadjusted primary endpoint by treatment group, FIG. 2A showing data for the subjects that completed the study (n=52) and FIG. 2B showing data for all subjects including dropouts (n=62).

FIGS. 3A-3B show data for parathyroid hormone (PTH) levels in the treatment groups, where FIG. 3A is a boxplot of PTH measurements per group and FIG. 3B shows a smoothed longitudinal summary of all PTH measurements per group. All measurements were log 2 transformed. Note the different y-axis scales.

FIGS. 4A-4B show data for FGF23 in the treatment groups, where FIG. 4A is a boxplot of FGF23 measurements per group and FIG. 4B shows a smoothed longitudinal summary of all FGF23 measurements per group. All measurements were log 2 transformed. Note the different y-axis scales.

FIGS. 5A-5B show data for calcium measurements per group, where FIG. 5A is a boxplot of calcium measurements per group and FIG. 5B shows a smoothed longitudinal summary of all calcium measurements per group. Note the different y-axis scales.

FIGS. 6A-6B shows phosphate data for in the treatment groups, where FIG. 6A is a boxplot of phosphate measurements per group and FIG. 6B is a smoothed longitudinal summary of all phosphate measurements per group. Note the different y-axis scales.

### DETAILED DESCRIPTION

### I. Definitions

Various aspects now will be described more fully hereinafter. Such aspects may, however, be embodied in many different forms and should not be construed as limited to the embodiments set forth herein; rather, these embodiments are provided so that this disclosure will be thorough and complete, and will fully convey its scope to those skilled in the art.

Where a range of values is provided, it is intended that each intervening value between the upper and lower limit of that range and any other stated or intervening value in that stated range is encompassed within the disclosure. For example, if a range of 1 μm to 8 μm is stated, it is intended that 2 μm, 3 μm, 4 μm, 5 μm, 6 μm, and 7 μm are also explicitly disclosed, as well as the range of values greater than or equal to 1 μm and the range of values less than or equal to 8 μm.

The singular forms “a,” “an,” and “the” include plural referents unless the context clearly dictates otherwise.

The word “about” when immediately preceding a numerical value means a range of plus or minus 10% of that value, e.g., “about 50” means 45 to 55, “about 25,000” means 22,500 to 27,500, etc., unless the context of the disclosure indicates otherwise, or is inconsistent with such an interpretation. For example, in a list of numerical values such as “about 49, about 50, about 55, “about 50” means a range extending to less than half the interval(s) between the preceding and subsequent values, e.g., more than 49.5 to less than 52.5. Furthermore, the phrases “less than about” a value or “greater than about” a value should be understood in view of the definition of the term “about” provided herein.

All percentages, parts and ratios are based upon the total weight of the topical compositions and all measurements made are at about 25° C., unless otherwise specified.

The terms “chronic hemodialysis”, “maintenance hemodialysis”, or ‘chronic maintenance hemodialysis” intend a hemodialysis regimen of at least two hemodialysis sessions per week, the hemodialysis session of any duration and at any location (e.g., in-center, outpatient, hospital, satellite, home) and of any dialysis modality (e.g., hemodiafiltration (HDF), slow, low-efficiency daily dialysis, continuous, veno-venous high-flux HDF). In one embodiment, “chronic hemodialysis” or “maintenance hemodialysis” intend a hemodialysis regimen of at least three hemodialysis sessions per week, the hemodialysis session of any duration and at any location and of any modality.

“Etelcalcetide” refers to the compound (2R)-3-[[(2S)-2-acetamido-3-[[(2R)-1-[[(2R) [[(2R)-1-[[(2R)-1-[[(2R)-1-[[(2R)-1-amino-5-(diaminomethydeneamino)-1-oxopentan yl]amino]-1-oxopropan-2-yl]amino]-5-(diaminomethydeneamino)-1-oxopentan-2-yl]amino]-5-(diaminomethydeneamino)-1-oxopentan-2-yl]amino]-5-(diaminomethydeneamino)-1-oxopentan-2-yl]amino]-1-oxopropan-2-yl]amino]-3-oxopropyl]disulfanyl]-2-aminopropanoic acid, including its pharmaceutically acceptable salts, such as the hydrochloride salt. Etelcalcetide is described in WO2014/210489, incorporated by reference herein.

“Parenterally” or “parenteral” intends a route of administration other than the mouth and alimentary canal, and includes subcutaneous, intramuscular, intravenous, intrathecal, intracisternal, intraarterial, intraspinal, and intraepidural.

The phrase “pharmaceutically acceptable” is employed herein to refer to those compounds, salts, compositions, dosage forms, etc., which are—within the scope of sound medical judgment—suitable for use in contact with the tissues of human beings and/or other mammals without excessive toxicity, irritation, allergic response, or other problem or complication, commensurate with a reasonable benefit/risk ratio. In some aspects, “pharmaceutically acceptable” means approved by a regulatory agency of the federal or a state government, or listed in the U.S. Pharmacopeia or other generally recognized pharmacopeia for use in mammals (e.g., animals), and more particularly, in humans.

“Pre-dialysis” intends a clinical state of impairment of kidney function that is expected to lead to either death or inclusion in kidney replacement therapy, such as dialysis and/or transplantation. In an embodiment, a pre-dialysis subject may have stage 3b or stage 4 chronic kidney disease.

By reserving the right to proviso out or exclude any individual members of any such group, including any sub-ranges or combinations of sub-ranges within the group, that can be claimed according to a range or in any similar manner, less than the full measure of this disclosure can be claimed for any reason. Further, by reserving the right to proviso out or exclude any individual substituents, analogs, compounds, ligands, structures, or groups thereof, or any members of a claimed group, less than the full measure of this disclosure can be claimed for any reason.

Throughout this disclosure, various patents, patent applications and publications are referenced. The disclosures of these patents, patent applications and publications in their entireties are incorporated into this disclosure by reference in order to more fully describe the state of the art as known to those skilled therein as of the date of this disclosure. This disclosure will govern in the instance that there is any inconsistency between the patents, patent applications and publications cited and this disclosure.

For convenience, certain terms employed in the specification, examples and claims are collected here. Unless defined otherwise, all technical and scientific terms used in this disclosure have the same meanings as commonly understood by one of ordinary skill in the art to which this disclosure belongs.

### II. Methods of Treatment

Methods for treating left ventricle hypertrophy (LVH) and/or for alleviating progression of LVH in subjects are provided. Methods for preventing LVH and for preventing progression of LVH are also provided. The methods comprise administering etelcalcetide, and in some embodiments, administering etelcalcetide prior to, during, or subsequent to dialysis. In an embodiment, etelcalcetide is administered parenterally. In one embodiment, etelcalcetide is administered intravenously. In other embodiments, etelacalcetide is administered intravenously at the end of or subsequent to hemodialysis. In another embodiment, etelcalcetide is administered subcutaneously.

Studies conducted in support of these methods will now be described with reference to Example 1.

A study was designed, as described in Example 1, to treat human subjects with either etelcalcetide or alfacalcidol, which have differing influences on serum levels of FGF23. Etelcalcetide, a calcimimetic drug, decreases in vivo blood FGF23 levels; whereas, alfacalcidol, a vitamin D hormone analog, increases in vivo blood FGF23 levels. During the course of the study, serum FGF23 and other biomarker levels were monitored and cardiac structure was assessed via cMRI. Using cMRI left ventricular mass was quantified.

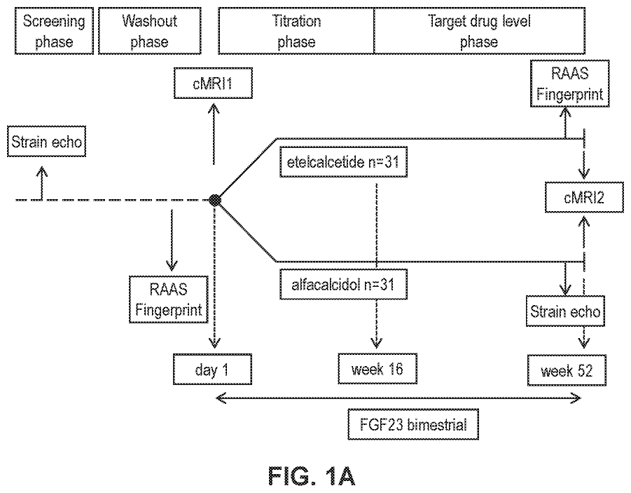

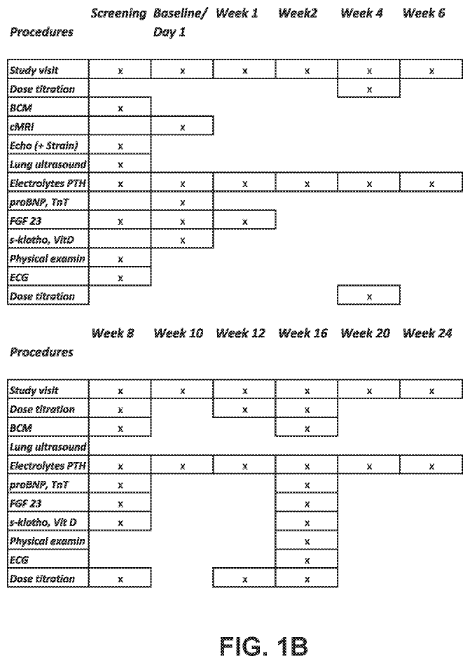

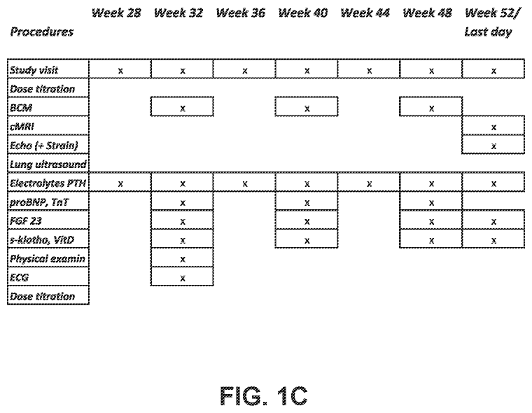

For the study, 62 patients were recruited, of which 30 patients were randomized to treatment with alfacalcidol and 32 to treatment with etelcalcetide. During the course of the 12-month study, ten patients dropped out during follow-up. Data for the main outcome (left ventricular mass index, LVMI in g/m²) was available for the patients at baseline (enrollment) and at the 1-year follow-up, except for three patients as noted in Example 1. FIG. 1A illustrates a flow chart of the study design.

Etelcalcetide causes a rapid, dose-dependent decrease in circulating levels of PTH, FGF23, calcium and phosphorus in CKD patients. A single intravenous dose of etelcalcetide can lower serum levels of PTH for up to 72 hours in patients on hemodialysis. FGF23 levels decrease by over 30% at 24 hours after a single 10 mg dose of etelcalcetide, while little or no effect is shown on 1.25(OH)₂ vitamin D levels (Martin, K. J. et al., Nephrol Dial Transplant, 2014: p. 29:385-92). Etelcalcetide dosage is, in one embodiment, between about 2.5 mg and about 15 mg, 3 times a week. In this study, the starting dose was 5 mg 3 times a week. To achieve a target value of PTH (100-300 pg/mL), the dosage was adapted every 4 weeks in steps of 2.5 mg or 5 mg during the titration phase (see FIG. 1A). Serum calcium were measured at every dialysis session, with a target levels of serum calcium corrected for serum albumin of ≥2.08 mmol/L.

Alfacalcidol is an analogue of vitamin D3 and can decrease PTH levels by ≥30% and increase FGF23 levels threefold (Hansen, R., et al., Nephrol Dial Transplant, 2012: p. 27:2263-9). The starting dose of alfacalcidol was 1 μg, administered as an intravenous bolus 3 times a week at the end of hemodialysis. Alfacalcidol dosage was at least 0.5 μg 3 times a week with no maximal dose. Titration was performed in 0.5-1 μg steps in 4 weeks intervals, depending on PTH values, serum calcium and phosphate levels. The target value of PTH was equivalent to the etelcalcetide group. Serum calcium corrected for serum albumin was no higher than 2.55 mmol/L and serum phosphate level was below 2.5 mmol/L.

A goal was to achieve a similar reduction in PTH in both study groups while FGF23 was elevated in the alfacalcidol arm and suppressed in the etelcalcetide arm in order to analyze the causality of FGF23 reduction on LVH and fibrosis. However, it is likely that the levels of PTH will vary, simply due to the different pharmacodynamics of the two drugs. Even though the dose of the study medication can be changed during the drug titration period as well as later on when necessary in order to reach target PTH levels, these adaptations are often limited by serum calcium and phosphate levels.

The timeline of the planned procedures, study visits and scheduled dose titrations of the study of Example 1 is visualized in FIGS. 1B-1C.

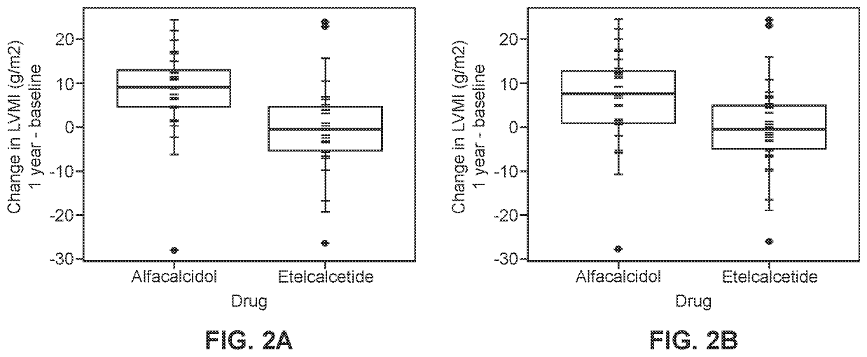

Turning now to FIGS. 2A-2B, data from the study is presented. In this data set, a per-protocol analysis was done where only patients who successfully completed the trial as planned per protocol were included in the data analysis. Thus, the 10 patients who dropped out during the study are removed from the analysis. FIG. 2A is a boxplot of the unadjusted primary endpoint for the alfacalcidol and the etelcalcetide treatment groups. The boxplot shows the primary study endpoint, difference of LVMI (g/m²) at 1 year and LVMI at baseline, for the 52 patients who completed the study. The unadjusted comparison of the primary endpoint by a 2-sample equal variances t-test yields a significant difference in LVMI change between the treatment groups (p=0.007156). Due to the non-normal distribution of the data, a non-parametric 2-sample Wilcoxon test was also performed to confirm the result (p=0.0006661).

An analysis adjusted for the stratification of the randomization was conducted using an Analysis of Covariance (ANCOVA) with outcome variable “Change of LVMI” and using the randomization stratification factors (residual kidney function, center of recruitment) as well as the LVMI at baseline as covariates. This resulted in an estimated adjusted mean difference in the change of LVMI of −8.2 (95% confidence interval −13.99 to −2.39) g/m² in the etelcalcetide group compared to the alfacalcidol group. The associated p-value was 0.00809, in accordance with the result of the unadjusted analysis.

In another analysis of the data, referred to as in intention-to-treat analysis, all patients who were randomized for the study are included in the data analysis. Thus, for the 10 patients who dropped out during the study all available measurements are used at the time they were taken. Out of those, for the 3 patients without MRI measurements the outcome values are imputed (obtained as mean values from patients of the same medication group with stratification factors identical to the patient with missing data). This data is shown in FIG. 2B.

Final data on the primary study endpoint, difference of LVMI (g/m²) at 1 year and LVMI at baseline, for the 62 patients who were randomized for the study is shown in FIG. 2B. The unadjusted comparison of the primary endpoint by a 2-sample equal variances t-test yields a significant difference in LVMI change between the treatment groups (p=0.04691). However, due to the distribution of the data the non-parametric Wilcoxon test is deemed more reliable and confirms the results (p=0.006098). The analysis by ANCOVA resulted in an estimated adjusted mean difference in the change of LVMI of −6.2 (95% confidence interval −11.72 to −0.71) g/m² in the etelcalcetide group compared to the alfacalcidol group. The associated p-value was 0.031, in accordance with the result of the unadjusted analysis.

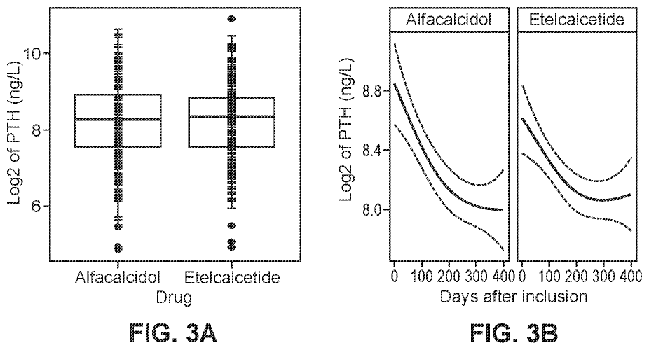

Turning now to FIGS. 3-6, data for the secondary endpoints of the study is presented. The data in the graphs of FIGS. 3-6 are based on measurements for all individuals with available visit dates (55 individuals). In FIGS. 3A-3B, the parathyroid hormone levels of the subjects in each treatment group is shown. On average, 17 measurements were available throughout follow-up for each individual. Overall measurements and longitudinal behavior was similar between groups.

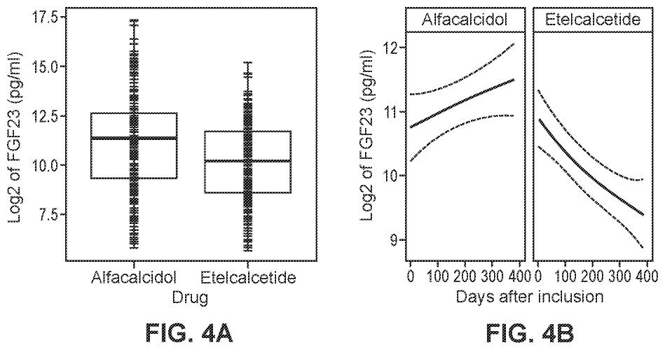

In FIGS. 4A-4B, the data on FGF23 blood levels for the subjects in each treatment group is shown. On average, 8 measurements were available throughout follow-up for each individual. Overall measurements and longitudinal behavior differed between groups, with lower values which were decreasing over time in the etelcalcetide group.

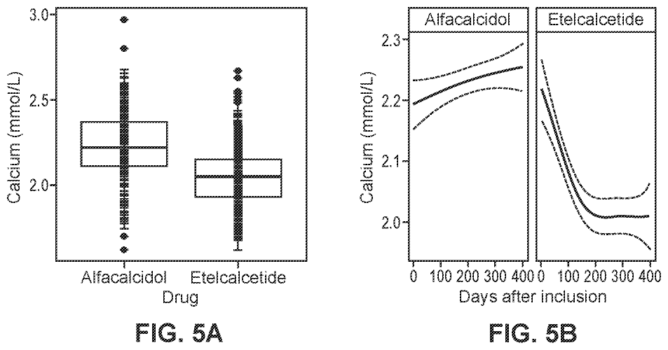

In FIGS. 5A-5B, the calcium blood levels for the subjects in each treatment group is shown. On average, 17 measurements were available throughout follow-up for each individual. Overall measurements and longitudinal differed between groups, with lower values which were decreasing over time in the etelcalcetide group.

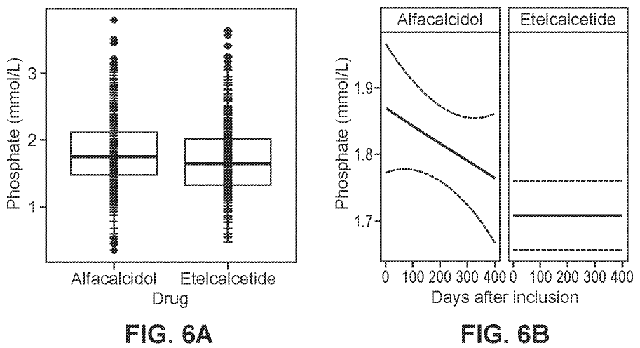

In FIGS. 6A-6B, the phosphate blood levels for the subjects in each treatment group is shown. On average, 17 measurements were available throughout follow-up for each individual. Overall measurements and longitudinal differed only slightly between groups.

The data shown above reveals the primary endpoint indicates a significant difference between the two treatment groups. The data shows that etelcalcetide effectively ameliorates LVH and cardiac fibrosis through a suppression of FGF23. These effects reduce the risk of and rate of cardiac death in patients under maintenance hemodialysis.

Accordingly, a method for treating, preventing and/or preventing or delaying progression of LVH is provided by administering etelcalcetide. The treatment is particularly beneficial to subjects on chronic or maintenance hemodialysis. That is, subjects receiving hemodialysis about two times per week or about three times per week. Subjects on chronic or maintenance hemodialysis for at least about 3 months, 4 months, 5 months, 6 months, 9 months or 12 months are considered candidates for the methods.

The data shows that treatment with etelcalcetide alleviates progression, slows progression, and/or delays progression of LVH by a median of about 6.0 g/m², about 6.3 g/m², about 6.7 g/m² or about 7.0 g/m² relative to left ventricle hypertrophy before treatment with etelcalcetide.

The data also shows that etelcalcetide is beneficial to mediate cardiac remodeling in a subject with LVH and on chronic dialysis. Administering etelcalcetide, for example, intravenously subsequent to hemodialysis, for at least approximately 12 months mediates cardiac remodeling. The cardiac remodeling is apparent, for example, by a reduction in LVH as determined by a cMRI assessment of left ventricular mass index by a median of about 6.0 g/m², about 6.3 g/m², about 6.7 g/m² or about 7.0 g/m² relative to left ventricle hypertrophy mass index in a population of subjects on maintenance hemodialysis and with left ventricle hypertrophy not yet treated with etelcalcetide.

Subjects contemplated for treatment are those with secondary hyperparathyroidism (SHPT) and/or those with a parathyroid hormone (PTH) level of greater than or equal to 300 pg/mL prior to administering etelcalcetide. In other embodiments, subjects contemplated for treatment with etelcalcetide are persons on maintenance dialysis or persons that are pre-dialysis. A pre-dialysis subject intends a person with impairment of kidney function that is clinically expected to lead to either death or inclusion in kidney replacement therapy, such as dialysis and/or transplantation. For example, the subject may have stage 3 or stage 4 chronic kidney disease (e.g., a glomerular filtration rate (GFR) of 45-49 mL/min (Stage 3A), or 30-44 mL/min (Stage 3B) or of 15-29 mL/min (Stage 4)) and not yet on maintenance dialysis. Treatment with etelcalcetide parenterally to subjects that are pre-dialysis but with an impaired kidney function is contemplated. LVH can develop in patients with a GFR of below 60 mL/min/1.73 m², (Di Lullo et al. Cardiorenal Med., 5(4): 254-266 (2015)), which is a GFR rate (or stage) where maintenance dialysis is not needed (e.g., the subject is pre-dialysis), and treatment with etelcalcetide to such patients is contemplated, for delaying progression of, treating, or ameliorating LVH. With regard to treatment of subjects on maintenance dialysis, the dialysis can be hemodialysis or peritoneal dialysis. In another embodiment, subjects contemplated for treatment are post-renal transplant subjects.

The dose of etelcalcetide is, in an embodiment, one that provides a blood parathyroid hormone (PTH) level of less than about 300 pg/mL. In another embodiment, the dose of etelcalcetide is one that provides a blood parathyroid hormone (PTH) level of between about 100 pg/mL to about 300 pg/mL.

The data also suggests a slowed development of cardiac fibrosis. Accordingly, a method to improve cardiac function in a subject with left ventricle hypertrophy and on maintenance dialysis is contemplated, by reducing circulating blood FGF23 levels in the subject for at least about 6 months, or 9 months, or 12 months by administering etelcalcetide subsequent to hemodialysis for at least about 6 months, or 9 months, or 12 months. As can be appreciated, this will reduce likelihood of cardiac death in a subject with left ventricle hypertrophy on maintenance dialysis.

The data shows that treatment with etelcalcetide for 6 months, 12 months, 18 months, or 24 months prevented LVMI progression, and also decreased FGF23 levels in dialysis patients with sHPT, compared to treatment with alfacalcidol. To date, no treatment for LVH progression in CKD patients has shown efficacy to mitigate cardiac remodeling. LVH remains a main contributor to the increased cardiovascular mortality in these patients. Prevention of LVH progression by approximately 6-8% is a proof for a clinically relevant reduction of myocardial remodeling. In addition to FGF23 mediated effects on the myocardium, reduced calcium levels in patients treated with etelcalcetide may reduce vascular calcification that by itself is associated with an increased risk of LVH and cardiovascular events in dialysis patients.

### III. Examples

The following examples are illustrative in nature and are in no way intended to be limiting.

### Example 1

### Treatment with Etelcalcetide or Alfacalcidol in Hemodialysis Patients with SHPT

Study Overview: A single blinded randomized trial of twelve months to test the effects of etelcalcetide compared to alfacalcidol on LVH and cardiac fibrosis in maintenance hemodialysis patients with secondary hyperparathyroidism (SHPT) was conducted. Both treatment regimens were titrated to equally suppress SHPT. Patients treated three times weekly with hemodialysis for between about 3 months and about 3 years and with parathyroid hormone (PTH) levels ≥300 pg/ml and LVH were enrolled.

The primary study endpoint was left ventricular mass index (LVMI) determined in g/m² at baseline and at 12 months by cardiac MRI (cMRI). Sample size calculation showed that 62 equally randomized patients would be necessary to detect a difference in LVMI of at least 20 g/m² between the two groups at 12 months. Due to the strong association of volume overload and LVH, randomization was additionally stratified by residual kidney function and regular body composition monitoring was performed to control patients' volume status.

Study medication was administered by the dialysis nurses intravenously after every hemodialysis session.

Secondary study endpoints were cardiac parameters measured by echocardiography, biomarker concentrations of bone metabolism (FGF23, vitamin D, PTH, calcium, phosphate, s-klotho), cardiac markers (proBNP, pre- and post-dialysis troponin T) and metabolites of the renin-angiotensin-aldosterone cascade (Ang I, Ang II, Ang 1-7, Ang 1-5, Ang 1-9, Aldosterone).

Study design: The study flow chart is presented in FIG. 1 and in Table 1. Following signed informed consent, patients were screened for LVH (i.e. interventricular septum thickness of ≥12 mm) and cardiac fibrosis using strain echocardiography. Volume status and fluid composition was explored by body composition monitoring (BCM) and lung ultrasound. Only patients who achieve euvolemia were eligible for enrollment to the study. All patients that were already treated with a calcimimetic drug or vitamin D therapy underwent a 4-week long washout phase in which the treatment will be discontinued.

Study participants who qualified for the study were centrally randomized at a 1:1 ratio to the following groups: a) etelcalcetide, b) alfacalcidol. Randomization was performed by a computer algorithm (www.meduniwien.ac.at/randomizer/web) and was stratified by residual kidney function, defined as an amount of 500 mL or more urine volume per day and the center where patients are recruited. To ensure that comparison groups were of approximately the same size and balanced in each center a block randomization (block size of 4) of anuria vs. residual renal function groups were used.

TABLE 1
Study design

| | Study phase | | | | | | |
|---|---|---|---|---|---|---|---|
| | | | | | Dose | Target | |
| | | Washout | | | titration | drug level | |
| | Screening | 4 weeks | Baseline | Allocation | 16 weeks | 36 weeks | Closeout |
| | Eligibility | If Vit D or | | | | | |
| | screen, | calcimimetic | | | | | |
| | Informed | in therapy | | | | | |
| | consent | | | | | | |
| Randomization | | | | X | | | |
| Laboratory analysis | X | | | | 2-4 week- | 4 week | |
| | | | | | intervals | intervals | |
| Strain Echo | X | | | | | | X |
| Lung ultrasound | X | | | | | | |
| BCM | X | | | | | 8 week | X |
| | | | | | | intervals | |
| cMRI | | | X | | | | X |
| INTERVENTIONS | | | | | | | |

| | | |
|---|---|---|
| Etelcalcetide | n = 50% | [symbol] |
| Alfacalcidol | n = 50% | [symbol] |

Treatment phase: The treatment phase started with a dose-titration phase of 16 weeks. Subjects were considered for dose titration of the investigational product every 4 weeks. Dose adjustment was based upon PTH values, serum electrolytes and safety assessment. Study visits took place in two-week intervals during the first 10 weeks of treatment followed by study visits every 4 weeks. The duration of the treatment phase was twelve months.

Study endpoints: The primary endpoint was the change of LVMI (quantified in g/m²) from baseline to months 12 between etelcalcetide and alfacalcidol assessed by cMRI. Secondary endpoints were the change in left atrial diameter LAD (mm), the change in LVMI- and LAD progression (%), the difference in cardiac fibrosis and fibrosis progression as measured with non-contrast T1 mapping (ms) and differences in cardiac function (ejection fraction %) as well as wall motion abnormalities (% change) as measured by cMRI and strain echocardiography after a year-long treatment with either drug. Other secondary objectives included changes in serum levels of FGF23, s-klotho, PTH, 25-OH-Vit-D and 1,25-(OH)2-Vit-D, phosphate, calcium, proBNP, pre- and postdialysis TnT and the metabolites of the RAAS cascade (Ang I, Ang II, Ang 1-7, Ang 1-5, Ang 1-9, Aldosterone) under either treatment as well as their association with the mentioned cardiac changes.

Outcomes measurements: cMRI: Two cardiac MRIs were planned for each patient. The baseline MRI took place before randomization, the second MRI took place after completing 12 months of treatment. Both were carried on the dialysis free day. The cMRI was analyzed by one radiologist blinded for the treatment allocation. Non-contrast cMRI was carried out using a 1.5 Tesla magnetic resonance imaging scanner (Siemens Avanto 1.5T, Siemens, Erlangen, Germany). Axial black-blood imaging was performed for visualization of cardiac anatomy. For the assessment of cardiac function, left ventricular muscle mass, and the visualization of possible wall motion abnormalities, multislice-multiphase cine imaging was performed in the long horizontal axis as well as in the short axis view through the entire heart. The ejection fraction (in percent) of both the left as well as the right ventricle were calculated in a semi-automatic fashion using dedicated software (Siemens Argus) based on the short-axis views. For assessment of cardiac function, the end-diastolic and end-systolic volume (in milliliter) was assessed in a semi-automatic fashion and the left ventricular muscle mass was calculated (Patel R. K. et al., Clin J Am Soc Nephrol., 2009: p. 4:1477-1483). The upper limit of normal left ventricular mass indexed for body surface area (LVM/BSA) values was considered to be 84.1 g/m² for male and 76.4 g/m² for female subjects (Salerno, M. P. et al., Transplantation Proceedings, 2013: p. 45:2660-2).

For the detection of myocardial fibrosis, fat-suppressed T2 weighted edema-sensitive imaging was performed. Non-contrast T1 mapping was performed to detect diffuse fibrotic processes (T1 time is measured in ms; measurement was conducted global, septal and nonseptal). The native myocardial T1 relaxation was a surrogate of myocardial fibrosis (Sparrow, P. et al., American Journal of Roentgenology, 2006: p. 187:630-5). In hemodialysis patients the interventricular septum is prone to the development of fibrosis in hemodialysis patients (Graham-Brown M. P. M. et al., Kidney International, 2016: p. 90:835-44).

Outcomes measurements: Strain echocardiography: Echocardiography for the evaluation of LVH was done during screening as well as at the end of the treatment phase. Doppler imaging or 2-dimensional speckle tracking echocardiography was used to measure strain and strain rate. With these techniques subclinical heart disease in fibrotic processes can be detected, with the predominant planes of strain initially affected mirroring the histological location of early fibrosis (Haland, T. F., et al., European Heart Journal of Cardiovascular Imaging, 2016: p. 17:613-21; C. Jellies, J. M et al., Journal of the American College of Cardiology, 2010: p. 56:89-97). Global longitudinal strain (GLS) was measured in % and it correlates with myocardial fibrosis (Saito, M, O. H et al., Eur Heart J Cardiovasc Imaging, 2012: p. 13:617-23). The physician performing the examination was blinded for the patient's treatment assignment.

Outcomes measurements: Body composition monitoring: BCM was performed during screening and was repeated in two-months intervals. BCM measurements were based on bioimpedance spectroscopy. The measurements were fed into a model to measure overhydration of an individual. Fluid overload assessed by BCM was expressed as an absolute value in liters or as a relative value in %. It is a reproducible body fluid volume determination over a wide range of body compositions in different states of health and disease. Only patients who achieved their optimal dry weight at the end of dialysis treatment and tolerated it well were enrolled in the study.

Outcomes measurements: Lung ultrasound: Extravascular lung water assessment was conducted as part of the screening procedures with the help of lung ultrasound, which can visualize lung edema and classify it semi-quantitatively. Only patients without signs of pulmonary edema were enrolled in the study.

Outcomes measurements: Laboratory analyses: Biochemical data was collected prior to hemodialysis at baseline and periodically (e.g., intact PTH, calcium, phosphate, 25-OH-Vit-D, 1,25-(OH)2-Vit-D every two weeks during the first 10 weeks followed by measurements every 4 weeks; while intact FGF23, s-klotho, proBNP and pre+postdialysis TnT was measured in 8-week-intervals). Additionally, a RAAS fingerprint was conducted before start and at the end of the treatment phase. The RAAS fingerprint is a mass spectrometry-based quantification of angiotensin metabolites. Serum samples were used to measure the following parameters: Ang I, Ang II, Ang 1-7, Ang 1-5, Ang 1-9, Aldosterone.

Intact PTH, calcium and phosphate were analyzed in serum samples using the Cobas assay (Roche, reference range of PTH: 15-65 pg/mL, calcium: 2.15-2.55 mmol/L, phosphate: 0.81-1.45 mmol/L). Vitamin D was measured using serum samples and a chemiluminescent-immunoassays (Diasorin, reference range of 1.25-(OH)2-VitD: 19.9-79.3 pg/mL; 25-OH-Vit-D 75-250 nmol/L). Ionized calcium was measured during every dialysis session (using blood gas analysis (ABL 800 Flex, Drott)). Intact FGF23 was analyzed in plasma samples using chemiluminescent—Immunoassays (DiaSorin, reference range 23.2-95.4 pg/mL). TnT and proBNP were measured from serum samples using COBAS electrochemiluminescence—immunoassays (Roche, reference range of TnT: 0-14 ng/L; reference range of proBNP 0-125 pg/mL).

The timeline of the planned procedures, study visits and scheduled dose titrations is visualized in FIGS. 1B-1C.

Investigational drugs: The etelcalcetide starting dose was 5 mg 3 times a week. To achieve a target value of PTH (100-300 pg/mL), the dosage was adapted every 4 weeks in steps of 2.5 mg or 5 mg during the titration phase. Serum calcium was measured at every dialysis session. Target levels of serum calcium corrected for serum albumin were ≥2.08 mmol/.

The alfacalcidol starting dose was 1 μg, administered as an intravenous bolus 3 times a week at the end of hemodialysis. Alfacalcidol dosage was at least 0.5 μg 3 times a week with no maximal dose. Titration was performed in 0.5-1 μg steps in 4 weeks intervals, depending on PTH values, serum calcium and phosphate levels. The target value of PTH was equivalent to the etelcalcetide group. Serum calcium corrected for serum albumin was no higher than 2.55 mmol/L and serum phosphate level was below 2.5

Other HPT treatment: Cinacalcet treatment as well as oral and intravenous vitamin D therapy were discontinued during the washout phase of 4 weeks (see FIG. 1). Phosphate binder therapy was continued and was adapted depending on serum electrolytes during the treatment phase. There were no restrictions on calcium supplements, the dialysate calcium concentration, or the type or dose of phosphate binders prescribed. Participants randomized to ETL were permitted to receive additional vitamin D analogs as a rescue therapy only when the investigator found it necessary to protect participant safety.

Statistical methods: Data was described by means and standard deviation or median and interquartile range for continuous symmetric and skewed variables, respectively. Distributions of the analyzed parameters were visualized by boxplots and histograms. The primary endpoint (change in LVMI from baseline to final measurement) was analyzed by Analysis of Covariance. The main variable in the model which was tested was group status, which represents the difference in measurement values one year after baseline between the two treatments. Baseline values for each patient were used as a covariate in the model and the interaction between group status and baseline values was checked. Furthermore, to account for stratification during randomization, the stratification factors were also included in the model. The secondary endpoints (changes of FGF23, s-klotho, PTH, 25-OH-Vit-D, 1,25-(OH)2-Vit-D, proBNP, pre- and postdialysis TnT as well as RAAS metabolites) were analyzed analogously. All analyses were conducted according to the intention to-treat principle. Two-sided p-values lower than 0.05 indicated statistical significance.

While a number of exemplary aspects and embodiments have been discussed above, those of skill in the art will recognize certain modifications, permutations, additions and sub-combinations thereof. It is therefore intended that the following appended claims and claims hereafter introduced are interpreted to include all such modifications, permutations, additions and sub-combinations as are within their true spirit and scope.

It is claimed:

1. Etelcalcetide for use in a method for treating left ventricle hypertrophy in a subject, wherein the etelcalcetide alleviates progression of left ventricle hypertrophy in the subject.

2. The use of claim 1, wherein the subject is a subject on chronic hemodialysis or maintenance hemodialysis.

3. The use of claim 1 or claim 2, wherein the subject receives hemodialysis three times per week.

4. The use of any one of claims 1-3, wherein the subject has received hemodialysis three times per week for at least about 3 months.

5. The use of claim 1, wherein the subject is a pre-dialysis subject.

6. The use of any one of claims 1-5, wherein etelcalcetide is formulated for parenteral administration.

7. The use of any one of claims 1-6, wherein etelcalcetide is formulated for intravenous administration.

8. The use of any one of claims 1-7, wherein etelcalcetide when provided to the subject for a period of 12 months is effective to alleviate progression of left ventricle hypertrophy by a median of about 6.7 g/m² relative to left ventricle hypertrophy before said administering.

9. The use of any one of claims 1-8, wherein the subject has secondary hyperparathyroidism (SHPT).

10. The use of any one of claims 1-9, wherein the subject has a parathyroid hormone (PTH) level of greater than or equal to 300 pg/mL prior to treatment with etelcalcetide.

11. The use of any one of claims 1-10, wherein etelcalcetide is at a dose that achieves a blood parathyroid hormone (PTH) level of less than about 300 pg/mL.

12. The use of any one of claims 1-10, wherein etelcalcetide is at a dose that achieves a blood parathyroid hormone (PTH) level of between about 100 pg/mL to about 300 pg/mL.

13. The use of any one of claims 1-12, wherein the etelcalcetide slows development of cardiac fibrosis in the subject.

14. The use of any one of claims 1-13, wherein the etelcalcetide alleviates progression of left ventricle hypertrophy in the subject, as measured by left ventricular mass index through cardiac magnetic resonance imaging (cMRI).

15. Etelcalcetide for use in a method for slowing progression of left ventricle hypertrophy in a subject on chronic hemodialysis and with left ventricle hypertrophy, wherein the etelcalcetide is formulated for intravenous administration.

16. The use of claim 15, wherein the etelcalcetide slows progression of left ventricle hypertrophy as measured by left ventricular mass index through cMRI by a median of about 6.7 g/m² relative to the subject's left ventricle hypertrophy mass index before said administering.

17. Use of etelcalcetide for the manufacture of a medicament for use in delaying progression of left ventricle hypertrophy in a subject on chronic hemodialysis and with left ventricle hypertrophy, wherein the medicament is formulated for intravenous administration and is prepared to be administered subsequent to hemodialysis for at least approximately 12 months.

18. The use of claim 17, whereby the medicament delays progression of left ventricle hypertrophy, as measured by left ventricular mass index through cMRI, by a median of about 6.7 g/m² relative to left ventricle hypertrophy mass index in a population of subjects on maintenance hemodialysis and with left ventricle hypertrophy not yet treated with etelcalcetide.

19. Use of etelcalcetide for the manufacture of a medicament for use in mediating cardiac remodeling in a subject with left ventricle hypertrophy and on chronic hemodialysis, wherein the medicament is formulated for intravenous administration and is prepared to be administered subsequent to hemodialysis for at least approximately 12 months.

20. The use of claim 19, whereby the medicament reduces left ventricle hypertrophy as determined by a cMRI assessment of left ventricular mass index by a median of about 6.7 g/m² relative to left ventricle hypertrophy mass index in a population of subjects on maintenance hemodialysis and with left ventricle hypertrophy not yet treated with etelcalcetide.

21. The use of any one of claims 15-20, wherein the subject receives hemodialysis approximately three times per week.

22. The use of any one of claims 15-21, wherein the subject has received hemodialysis approximately three times per week for at least about 3 months.

23. The use of any one of claims 15-22, wherein the subject has secondary hyperparathyroidism (SHPT).

24. The use of any one of claims 15-23, wherein the subject has a parathyroid hormone (PTH) level of greater than or equal to 300 pg/mL prior to treatment with the medicament.

25. The use of any one of claims 15-24, wherein the medicament is formulated for administration at a dose of etelcalcetide that provides a blood parathyroid hormone (PTH) level of less than about 300 pg/mL.

26. The use of any one of claims 15-24, wherein the medicament is formulated for administration at a dose of etelcalcetide that provides a blood parathyroid hormone (PTH) level of between about 100 pg/mL to about 300 pg/mL.

27. The use of any one of claims 15-26, wherein the use slows development of cardiac fibrosis.

28. Use of etelcalcetide for the manufacture of a medicament for improving cardiac function in a subject with left ventricle hypertrophy and on maintenance hemodialysis, wherein the medicament is formulated for intravenous administration and is prepared to be administered for at least about 6 months subsequent to hemodialysis to achieve reduction in circulating blood FGF23 levels in the subject for at least about 6 months.

29. The use of claim 28, wherein the medicament slows development of cardiac fibrosis as measured by T1 weighted cMRI.

30. The use of claim 28 or claim 29, wherein the medicament is prepared to be administered to the subject for at least about 9 months or at least about 12 months.

31. Use of etelcalcetide for the manufacture of a medicament to reduce likelihood of cardiac death in a subject with left ventricle hypertrophy on maintenance hemodialysis, wherein the medicament is formulated for intravenous administration and is prepared to be administered for at least about 6 months subsequent to hemodialysis to achieve reduction in circulating blood FGF23 levels in the subject for at least about 6 months.

32. The use of claim 31, wherein the medicament slows development of cardiac fibrosis as measured by T1 weighted cMRI.

33. The use of claim 31 or claim 32, wherein the medicament is prepared to be administered to the subject or at least about 9 months or at least about 12 months.
